(19) United States
(12) Patent Application Publication
(10) Pub. No.: US 2010/0249346 A1
(43) Pub. Date: Sep. 30, 2010

(54) METALLOCENE COMPOUNDS BASED ON ETHANEDIYL-BRIDGED INDENE AND CYCLOPENTADITHIOPHENE LIGANDS

(75) Inventors: Michael Schiendorfer, Frankfurt (DE); Luigi Resconi, Ferrara (IT); Eleonora Ciaccia, Ferrara (IT)

Correspondence Address: BASELL USA INC., NEWTOWN SQUARE CENTER, 3801 WEST CHESTER PIKE, BLDG. B, NEWTOWN SQUARE, PA, 19073, US

(73) Assignee: Basell Polyolefine GmbH, WEsseling (DE)

(21) Appl. No.: 12/734,171

(22) PCT Filed: Oct. 1, 2008
(86) PCT No.: PCT/EP2008/063138

§ 371 Date: Apr. 15, 2010

Related U.S. Application Data
(63) Continuation of application No. 61/000,936, filed on Oct. 30, 2007.

(30) Foreign Application Priority Data
Oct. 19, 2007 (EP) 07118921.1

(51) Int. Cl. C08F 4/52; C07D 495/04; B01J 31/14

(52) U.S. Cl. 526/160; 549/3; 502/103; 502/118; 549/43

(57) ABSTRACT

A metallocene compound of formula (I)

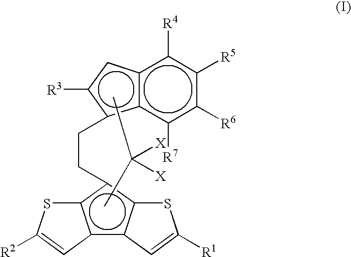

Wherein
M is an atom of a transition metal;
X, is a hydrogen atom, a halogen atom, or a hydrocarbon group optionally containing heteroatoms
R¹ and R², equal to or different from each other, are C₁-C₄₀ hydrocarbon radical optionally containing heteroatoms; R³ is a C₁-C₄₀ hydrocarbon radical optionally containing heteroatoms; R⁴, R⁵, R⁶ and R⁷, equal to or different from each other, are hydrogen atoms or C₁-C₄₀ hydrocarbon radical optionally containing heteroatoms or groups among R⁴, R⁵, R⁶ and R⁷ can also be joined to form a from 4 to 7 membered ring.

This application is the U.S. national phase of International Application PCT/EP2008/063138, filed Oct. 1, 2008, claiming priority to European Patent Application 07118921.1 filed Oct. 19, 2007, and the benefit under 35 U.S.C. 119(e) of U.S. Provisional Application No. 61/000,936, filed Oct. 30, 2007; the disclosures of International Application PCT/EP2008/063138, European Patent Application 07118921.1 and U.S. Provisional Application No. 61/000,936, each as filed, are incorporated herein by reference.

The present invention relates to a new class of ethylene bridged metallocene compounds wherein one ligand is a cyclopentadithiophene moiety and the other one is a substituted indenyl radical.

Metallocene compounds containing cyclopentadithiophene moieties are well known in the art. For example WO01/047939 discloses metallocene compounds containing cyclopentadithiophene moieties, however almost all the compounds exemplified has a silicon bridge. In Macromolecular Chemistry and Physics, 2005, 206, 1405-1438, among others, the following compounds have been tested:

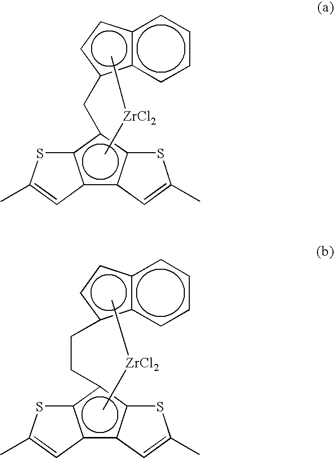

By comparing the polymerization activities of these two compounds (table 7) compound (a) has a considerably higher polymerization activity than compound (b).

However, even if silicon bridged compounds show a high polymerization activity the applicant discovered that this behavior can be reversed if the indenyl moiety is substituted.

The applicant unexpectedly found that the polymerization activity of this kind of metallocene compounds can be increased by using an ethylene radical as bridge and a substituted indenyl compound as one of the H-moiety.

An object of the present invention is therefore a metallocene compound of formula (I)

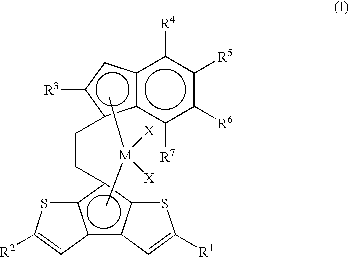

### Wherein

M is an atom of a transition metal selected from those belonging to group 3, 4, or to the lanthanide or actinide groups in the Periodic Table of the Elements; preferably M is zirconium, titanium or hafnium;
X, equal to or different from each other, is a hydrogen atom, a halogen atom, a R, OR, OSO₂CF₃, OCOR, SR, NR₂ or PR₂ group wherein R is a linear or branched, cyclic or acyclic, C₁-C₄₀-alkyl, C₂-C₄₀ alkenyl, C₂-C₄₀ alkynyl, C₆-C₄₀-aryl, C₇-C₄₀-alkylaryl or acyclic, C₁-C₄₀-alkyl, C₂-C₄₀ alkenyl, C₂-C₄₀ alkynyl, C₆-C₄₀-aryl, C₇-C₄₀-alkylaryl or C₇-C₄₀-arylalkyl radical; optionally containing heteroatoms belonging to groups 13-17 of the Periodic Table of the Elements; or two X groups can be joined together to form a group OR′O wherein R′ is a C₁-C₂₀-alkylidene, C₆-C₂₀-arylidene, C₇-C₂₀-alkylarylidene, or C₇-C₂₀-arylalkylidene radical; preferably X is a hydrogen atom, a halogen atom or R group; more preferably X is chlorine or a methyl radical;
R¹ and R², equal to or different from each other, are C₁-C₄₀ hydrocarbon radical optionally containing heteroatoms belonging to groups 13-17 of the Periodic Table of the Elements such as methyl or ethyl radical; preferably R¹ and R² are linear C₁-C₂₀-alkyl, such as a methyl, or ethyl radicals;
R³ is a C₁-C₄₀ hydrocarbon radical optionally containing silicon atoms, germanium atoms or heteroatoms belonging to groups 15-16 of the Periodic Table of the Elements; preferably R³ is a linear C₁-C₂₀-alkyl, such as a methyl, or ethyl radical;
R⁴, R⁵, R⁶ and R⁷, equal to or different from each other, are hydrogen atoms or C₁-C₄₀ hydrocarbon radical optionally containing silicon atoms, germanium atoms or heteroatoms belonging to groups 15-16 of the Periodic Table of the Elements or two groups among R⁴, R⁵, R⁶ and R⁷ can also be joined to form a from 4 to 7 membered ring, preferably a 5-6 membered ring that can be aliphatic or aromatic and can contain silicon atoms, Germanium atoms or heteroatoms belonging to groups 15-16 of the Periodic Table of the Elements; said ring can contain one or more C₁-C₁₀ hydrocarbon radicals as substituents.

Preferred classes of the compound of formula (I) have the following formulas (IIa), (IIb), (IIc) and (IId)

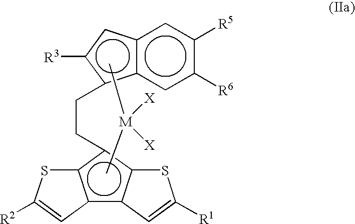

Wherein R¹, R², R³, R⁵, R⁶M and X have the above described meaning; preferably R⁵ and R⁶ are hydrogen atoms;

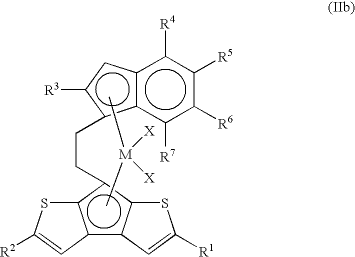

Wherein R¹, R², R³, R⁵, R⁶, M and X have the above described meaning; and R⁴ and R⁷, equal to or different from each other, are hydrogen atoms, C₁-C₂₀ alkyl, C₆-C₂₀-aryl, C₇-C₂₀-alkylaryl or C₇-C₂₀ᵣ arylalkyl radicals; with the proviso that at least one between R⁴ and R⁷ is different from hydrogen atom; preferably R⁴ and R⁷ are C₁-C₂₀ alkyl radicals, or R⁴ is a C₆-C₂₀-aryl or C₇-C₂₀-arylalkyl radical and R⁷ is a hydrogen atom;

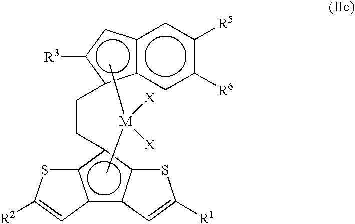

Wherein R¹, R², R³, M and X have the above meaning; and R⁵ and R⁶, equal to or different from each other, are C₁-C₂₀ alkyl radicals optionally containing silicon atoms, germanium atoms or heteroatoms belonging to groups 15-16 of the Periodic Table, or R⁵ and R⁶ can be joined to form a 5-6 membered ring, said ring can be aliphatic or aromatic and can contain silicon atoms, germanium atoms or heteroatoms belonging to groups 15-16 of the periodic table; said ring can further bear C¹-C¹⁰ alkyl substituents.

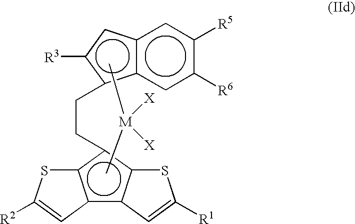

Wherein R¹, R², R³, R⁶, M and X have the above described meaning; and R⁴ and R⁷, equal to or different from each other, are C₁-C₂₀ alkyl, C₆-C₂₀-aryl, C₇-C₂₀-alkylaryl or C₇-C₂₀-arylalkyl radicals; preferably R⁴ and R⁷ are C₁-C₂₀ alkyl radicals; R⁶ is preferably hydrogen atom.

A further object of the present invention is a catalyst system for the polymerization of olefins obtainable by contacting:

a) a metallocene compound of formula (I);
b) at least an alumoxane or a compound able to form an alkylmetallocene cation; and
c) optionally an organo aluminum compound.

Preferably the metallocene compounds have formulas selected from (IIa), (IIb) or (IIc). Alumoxanes used as component b) in the catalyst system according to the present invention can be obtained by reacting water with an organo-aluminium compound of formula HⱼAlU₃₋ⱼ or HⱼAl₂U₆₋ⱼ, where the U substituents, same or different, are hydrogen atoms, halogen atoms, C₁-C₂₀-alkyl, C₃-C₂₀-cycloalkyl, C₆-C₂₀-aryl, C₇-C₂₀-alkylaryl or C₇-C₂₀-arylalkyl radicals, optionally containing silicon or germanium atoms, with the proviso that at least one U is different from halogen, and j ranges from 0 to 1, being also a non-integer number. In this reaction the molar ratio of Al/water is preferably comprised between 1:1 and 100:1. The alumoxanes used in the catalyst system according to the invention are considered to be linear, branched or cyclic compounds containing at least one group of the type:

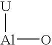

wherein the substituents U, same or different, are defined above.

In particular, alumoxanes of the formula:

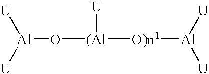

can be used in the case of linear compounds, wherein n¹ is 0 or an integer of from 1 to 40 and the substituents U are defined as above; or alumoxanes of the formula:

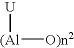

can be used in the case of cyclic compounds, wherein n² is an integer from 2 to 40 and the U substituents are defined as above.

Examples of alumoxanes suitable for use according to the present invention are methylalumoxane (MAO), tetra-(isobutyl)alumoxane (TIBAO), tetra-(2,4,4-trimethyl-pentyl)alumoxane (TIOAO), tetra-(2,3-dimethylbutyl)alumoxane (TDMBAO) and tetra-(2,3,3-trimethylbutyl)alumoxane (TTMBAO).

Particularly interesting cocatalysts are those described in WO 99/21899 and in WO01/21674 in which the alkyl and aryl groups have specific branched patterns.

Non-limiting examples of aluminium compounds that can be reacted with water to give suitable alumoxanes (b), described in WO 99/21899 and WO01/21674, are: tris(2,3,3-trimethyl-butyl)aluminium, tris(2,3-dimethyl-hexyl)aluminium, tris(2,3-dimethyl-butyl)aluminium, tris(2,3-dimethyl-pentyl)aluminium, tris(2,3-dimethyl-heptyl)aluminium, tris(2-methyl-3-ethyl-pentyl)aluminium, tris(2-methyl-3-ethyl-hexyl)aluminium, tris(2-methyl-3-ethyl-heptyl)aluminium, tris(2-methyl-3-propyl-hexyl)aluminium, tris(2-ethyl-3-methyl-butyl)aluminium, tris(2-ethyl-3-methyl-pentyl)aluminium, tris(2,3-diethyl-pentyl)aluminium, tris(2-propyl-3-methyl-butyl)aluminium, tris(2-isopropyl-3-methyl-butyl)aluminium, tris(2-isobutyl-3-methyl-pentyl)aluminium, tris(2,3,3-trimethyl-pentyl)aluminium, tris(2,3,3-trimethyl-hexyl)aluminium, tris(2-ethyl-3,3-dimethyl-butyl)aluminium, tris(2-ethyl-3,3-dimethyl-pentyl)aluminium, tris(2-isopropyl-3,3-dimethyl-butyl)aluminium, tris(2-trimethylsilyl-propyl)aluminium, tris(2-methyl-3-phenyl-butyl)aluminium, tris(2-ethyl-3-phenyl-butyl)aluminium, tris(2,3-dimethyl-3-phenyl-butyl)aluminium, tris(2-phenyl-propyl)aluminium, tris[2-(4-fluoro-phenyl)-propyl]aluminium, tris[2-(4-chloro-phenyl)-propyl]aluminium, tris[2-(3-isopropyl-phenyl)-propyl]aluminium, tris(2-phenyl-butyl)aluminium, tris(3-methyl-2-phenyl-butyl)aluminium, tris(2-phenyl-pentyl)aluminium, tris[2-(pentafluorophenyl)-propyl]aluminium, tris[2,2-diphenyl-ethyl]aluminium and tris[2-phenyl-2-methyl-propyl]aluminium, as well as the corresponding compounds wherein one of the hydrocarbyl groups is replaced by a hydrogen atom, and those wherein one or two of the hydrocarbyl groups are replaced with an isobutyl group.

Amongst the above aluminium compounds, trimethylaluminium (TMA), triisobutylaluminium (TIBA), tris(2,4,4-trimethyl-pentyl)aluminium (TIOA), tris(2,3-dimethylbutyl)aluminium (TDMBA) and tris(2,3,3-trimethylbutyl)aluminium (TTMBA) are preferred.

Non-limiting examples of compounds able to form an alkylmetallocene cation are compounds of formula D⁺E⁻, wherein D⁺ is a Brønsted acid, able to donate a proton and to react irreversibly with a substituent X of the metallocene of formula (I) and E⁻ is a compatible anion, which is able to stabilize the active catalytic species originating from the reaction of the two compounds, and which is sufficiently labile to be removed by an olefinic monomer. Preferably, the anion E⁻ comprises one or more boron atoms. More preferably, the anion E⁻ is an anion of the formula BAr₄⁽⁻⁾, wherein the substituents Ar, which can be identical or different, are aryl radicals such as phenyl, pentafluorophenyl or bis(trifluoromethyl)phenyl. Tetrakis-pentafluorophenyl borate is a particularly preferred compound, as described in WO 91/02012. Moreover, compounds of formula BAr₃ can be conveniently used. Compounds of this type are described, for example, in the international patent application WO 92/00333. Other examples of compounds able to form an alkylmetallocene cation are compounds of formula BAr₃P wherein P is a substituted or unsubstituted pyrrol radical. These compounds are described in WO01/62764. Compounds containing boron atoms can be conveniently supported according to the description of DE-A-19962814 and DE-A-19962910. All these compounds containing boron atoms can be used in a molar ratio between boron and the metal of the metallocene comprised between about 1:1 and about 10:1; preferably 1:1 and 2.1; more preferably about 1:1.

Non limiting examples of compounds of formula D⁺E⁻ are:
Tributylammonium tetrakis(pentafluorophenyl)borate,
Tributylammonium tetrakis(pentafluorophenyl)aluminate,
Tributylammonium tetrakis(trifluoromethylphenyl)borate,
Tributylammonium tetrakis(4-fluorophenyl)borate,
Dimethylbenzylammonium-tetrakis(pentafluorophenyl)borate,
Dimethylhexylammonium-tetrakis(pentafluorophenyl)borate,
N,N-Dimethylanilinium-tetrakis(pentafluorophenyl)borate,
N,N-Dimethyl anilinium-tetrakis(pentafluorophenyl)aluminate,
Di(iso-propyl)ammonium-tetrakis(pentafluorophenyl)borate,
Di(cyclohexyl)ammonium tetrakis(pentafluorophenyl)borate,
Triphenylcarbenium tetrakis(pentafluorophenyl)borate,
Triphenylcarbenium tetrakis(pentafluorophenyl)aluminate,
Ferrocenium tetrakis(pentafluorophenyl)borate,
Ferrocenium tetrakis(pentafluorophenyl)aluminate.

Organic aluminum compounds used as compound c) are those of formula HⱼAlU₃₋ⱼ or HⱼAl₂U₆₋ⱼ as described above.

The catalysts of the present invention can also be supported on an inert carrier. This is achieved by depositing the metallocene compound a) or the product of the reaction thereof with the component b), or the component b) and then the metallocene compound a) on an inert support. The support can be a porous solid such as talc, a sheet silicate, an inorganic oxide or a finely divided polymer powder (e.g. polyolefin). Suitable inorganic oxides may be found among the oxides of elements of groups 2, 3, 4, 5, 13, 14, 15 and 16 of the Periodic Table of the Elements. Examples of oxides preferred as supports include silicon dioxide, aluminum oxide, and also mixed oxides of the elements calcium, aluminum, silicon, magnesium or titanium and also corresponding oxide mixtures, magnesium halides, styrene/divinylbenzene copolymers, polyethylene or polypropylene. Other inorganic oxides which can be used alone or in combination with the above mentioned preferred oxidic supports are, for example, MgO, ZrO₂, TiO₂ or B₂O₃.

A suitable class of supports which can be used is that constituted by porous organic supports functionalized with groups having active hydrogen atoms. Particularly suitable are those in which the organic support is a partially crosslinked styrene polymer. Supports of this type are described in European application EP-633 272.

Another class of inert supports particularly suitable for use according to the invention is that of polyolefin porous prepolymers, particularly polyethylene.

A further suitable class of inert supports for use according to the invention is that of porous magnesium halides such as those described in international application WO 95/32995.

The support materials used preferably have a specific surface area in the range from 10 to 1 000 m²/g, a pore volume in the range from 0.1 to 5 ml/g and a mean particle size of from 1 to 500 μm. Preference is given to supports having a specific surface area in the range from 50 to 500 m²/g, a pore volume in the range from 0.5 to 3.5 ml/g and a mean particle size in the range from 5 to 350 μm. Particular preference is given to supports having a specific surface area in the range from 200 to 400 m²/g, a pore volume in the range from 0.8 to 3.0 ml/g and a mean particle size of from 10 to 300 μm.

The inorganic support can be subjected to a thermal treatment, e.g. to remove adsorbed water. Such a drying treatment is generally carried out at from 80 to 300° C., preferably from 100 to 200° C., with drying at from 100 to 200° C. preferably being carried out under reduced pressure and/or a blanket of inert gas (e.g. nitrogen), or the inorganic support can be calcined at from 200 to 1000° C. to produce the desired structure of the solid and/or set the desired OH concentration on the surface. The support can also be treated chemically using customary desiccants such as metal alkyls, preferably aluminum alkyls, chlorosilanes or SiCl₄, or else methylaluminoxane. Appropriate treatment methods are described, for example, in WO 00/31090.

The inorganic support material can also be chemically modified. For example, treatment of silica gel with (NH₄)₂SiF₆ leads to fluorination of the silica gel surface, or treatment of silica gels with silanes containing nitrogen-, fluorine- or sulfur-containing groups leads to correspondingly modified silica gel surfaces.

Organic support materials such as finely divided polyolefin powders (e.g. polyethylene, polypropylene or polystyrene) can also be used and are preferably likewise freed of adhering moisture, solvent residues or other impurities by means of appropriate purification and drying operations before use. It is also possible to use functionalized polymer supports, e.g. supports based on polystyrene, via whose functional groups, for example ammonium or hydroxy groups, at least one of the catalyst components can be immobilized. The solid compound obtained by supporting the catalyst system object of the present invention on a carrier in combination with the further addition of the alkylaluminium compound either as such or prereacted with water if necessary, can be usefully employed in the gas-phase or slurry polymerization.

The catalyst system of the present invention can be used also in a solution polymerization process.

For the purpose of the present invention the term “solution polymerization” means preferably that the polymer is fully soluble in the polymerization medium at the polymerization temperature used, and in a concentration range of at least 5% by weight; more preferably from 5 to 50% by weight.

In order to have the polymer completely soluble in the polymerization medium, a mixture of monomers for copolymers or only one monomer for homopolymers in the presence of an inert solvent can be used. This solvent can be an aliphatic or cycloaliphatic hydrocarbon such as hexane, heptane, isooctane, isododecane, cyclohexane and methylcyclohexane. It is also possible to use mineral spirit or a hydrogenated diesel oil fraction. Also aromatic hydrocarbons can be used such as toluene. Preferred solvents to be used are cyclohexane and methylcyclohexane. When propylene is used as monomer for the obtainment of propylene copolymers in solution polymerization process, the propylene content in the liquid phase of the polymerization medium preferably ranges from 5% to 60% by weight; more preferably from 20% to 50% by weight.

When olefins higher than propylene are present in a concentration above 50% wt of the total olefin content of the polymerization medium, the neat monomers can be used as the polymerization solvent, thus avoiding or minimizing the use of inert solvents.

The catalyst system comprising the metallocene compound of formula (I) can be used for polymerizing olefins, in particular alpha-olefins in high yields to give polymers having high molecular weight. Therefore a further object of the present invention is a process for preparing an alpha-olefin polymer comprising contacting under polymerization conditions one or more alpha-olefins of formula CH₂═CHA wherein A is hydrogen or a C₁-C₂₀ alkyl radical, in the presence of a catalyst system as described above.

Non limitative examples of alpha-olefins of formula CH₂═CHA are: ethylene, propylene, 1-butene, 1-hexene, 1-octene and 4-methyl-1-pentene, preferred alpha olefins are ethylene, propylene and 1-butene.

The metallocene compounds of formula (I), object of the present invention, are particularly suitable for the homo and copolymerization of propylene. In fact, the metallocene-based catalyst system of the present invention when used for homo or copolymerizing propylene are able to give polymers in high yields also at high temperatures rendering thus possible to use it in the industrial plants that use polymerization temperatures higher than 50° C. and that can be comprised between 60° and 200° C., preferably between 80° C. and 120° C.

The metallocene compounds of the present invention are also particularly suitable for the preparation of copolymers of ethylene and higher alpha olefins, such as propylene, 1-butene, 1-hexene, 1-octene. The copolymers have a comonomer content ranging from 5 to 50% by mol. Particularly preferred are ethylene/1-butene copolymer having a content of 1-butene derived units ranging from 5 to 50% by mol.

Further the metallocene compounds of formula (I), object of the present invention, are particularly suitable for the homo and copolymerization of 1-butene. In fact, the metallocene-based catalyst system of the present invention when used for homo or copolymerizing 1-butene are able to give polymers in high yields also at high temperatures rendering thus possible to use it in the industrial plants that use polymerization temperatures higher than 40° C. and that can be comprised between 50° and 100° C., preferably between 60 and 90° C. Therefore a further object of the present invention is a process for the preparation of 1-butene homo or copolymers comprising the step of contacting, under polymerization conditions, 1-butene and optionally ethylene, propylene or one or more alpha olefins of formula CH₂═CHA¹, wherein A¹ is a C₃-C₂₀ alkyl radical, in the presence of a catalyst system described above. This process is preferably carried out in solution in liquid monomer as described above.

Examples of alpha olefins of formula CH₂═CHA¹ are 1-hexene, 1-octene and 4-methyl-1-pentene, preferred alpha olefins are ethylene and propylene.

The polymerization pressure is generally comprised between 0.5 and 100 bar.

The polymerization yields depend on the purity of the metallocene compound of the catalyst. The metallocene compounds obtained by the process of the invention can therefore be used as such or can be subjected to purification treatments.

Further object of the present invention is a ligand of formula (III)

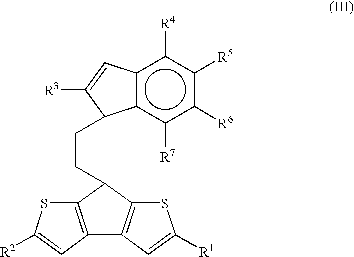

or its double bond isomer
wherein R¹, R², R³, R⁴R⁵, R⁶ and R⁷ have the meaning reported above.

Preferred ligands have formulas (IIIa), (IIIb) (IIIc) or (IIId):

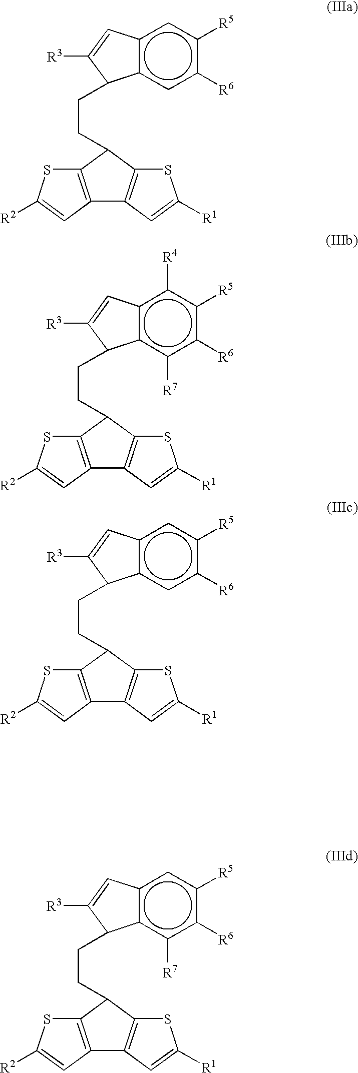

or their double bond isomers
wherein R¹, R², R³, R⁴, R⁵, R⁶ and R⁷ have the meaning reported above for the corresponding complex of formula (IIa), (IIb) (IIc) and (IId).

The metallocene compounds of formula (I) can be obtained with a process comprising the steps of reacting the dianion with a suitable transition metal source such as metal tetrahalide as for example zirconium tetrachloride. The dianion can be obtained for example by the deprotonation of the ligand of formula (III), for example by using an organolithium compound such as butyl or methyl lithium.

The above processes are preferably carried out in an aprotic solvent, either polar or apolar. Said aprotic solvent is preferably an aromatic or aliphatic hydrocarbon, optionally halogenated, or an ether; more preferably it is selected from benzene, toluene, pentane, hexane, heptane, cyclohexane, dichloromethane, diethylether, tetrahydrofurane and mixtures thereof. The above process is carried out at a temperature ranging from −100° C. to +80° C., more preferably from −20° C. to +70° C.

The ligand of formula (III) can be synthesized with a process comprising the following steps:

i) contacting the moiety of formula (IVa)

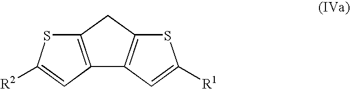

Wherein R¹ and R² have the meaning reported above
with a base selected from the group consisting of metallic sodium and potassium, sodium and potassium hydroxide and an organolithium compound, wherein the molar ratio between the compound of the formula (II) and said base is at least 1:1; preferably the organolithium compound has formula LiR^{a} wherein R^{a} is a C₁-C₄₀ hydrocarbon group, preferably R^{a} is a C₁-C₄₀-alkyl, C₆-C₄₀-aryl, C₇-C₄₀-alkylaryl or C₇-C₄₀-arylalkyl radical; more preferably R^{a} is a C₁-C₂₀-alkyl or a C₆-C₂₀-aryl radical; such as methyllithium, butyllithium or phenyllithium;
   ii) contacting the reaction product of step i) with a compound of formula (IVb)

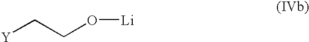

Wherein Y is a halogen atom, preferably Y is bromine or chlorine; the contact is preferably carried out by contacting a solution of the compound (IVb) with a solution/suspension of the reaction product of step i); after the reaction has been completed; preferably in a time ranging from 10 minutes to 3 hours a compound able to donate a proton, such as water or HCl is added to the reaction to form a compound of formula (IVc)

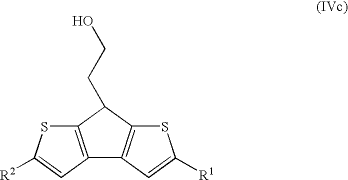

iii) contacting the compound of formula (IVc) with trifluoromethanesulfonic acid anhydride in the presence of a weak broensted base such as pyridine or a tertiary amine to form the product of formula (IVd)

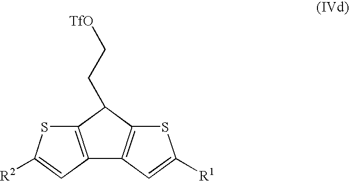

wherein OTf is the trifluoromethanicsulfonate residue;
   iv) contacting the product (IVd) with a salt of formula (IVe)

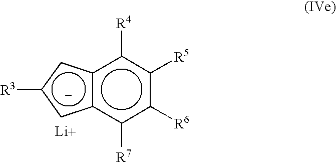

to obtain the final product.

All the above steps are preferably performed under inert atmosphere and carried out in an aprotic solvent, either polar or apolar. Said aprotic solvent is preferably an aromatic or aliphatic hydrocarbon, optionally halogenated, or an ether; more preferably it is selected from benzene, toluene, pentane, hexane, heptane, cyclohexane, dichloromethane, diethylether, tetrahydrofurane and mixtures thereof. The above process is carried out at a temperature ranging from −100° C. to +80° C., more preferably from −20° C. to +70° C.

The following examples are given to illustrate and not to limit the invention.

### EXAMPLES

### Preparation of Metallocene Compounds

### Ethylene-1-(2-methyl-indenyl)-2-(2,5-Dimethyl-cyclopenta[1,2-b;4,3-b′]dithiophene-7-yl)-ZrCl₂ (A1)

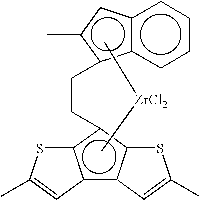

To 1.22 g (3.35 mmol) of 1-(2-methyl-inden-1-yl)-2-(2,5-dimethyl-cyclopenta[1,2-b;4,3-b′]dithiophen-7-yl)-ethane in 80 ml of dry diethylether were added 2.68 ml n-butyllithium (6.70 mmol/2.5M in hexane) under argon and ice cooling. The resulting white suspension was stirred for 3 h at rt. To this suspension was added a suspension of 0.78 g (3.35 mmol) of zirconiumtetrachloride in 10 ml of dry toluene. The yellow suspension was stirred over night at rt and filtered. The filter cake was washed four times with 20 ml of dry dichloromethane in each case. The combined redorange organic layers were concentrated and washed three times with 10 ml of dry diethylether in each case. After drying of the residue 1.03 g (1.96 mmol/59%) of an orange powder were obtained.

¹H-NMR (500.1 MHz, CD₂Cl₂, MIS 333): δ=2.37 (s, 3H, H-7), 2.41 (d, 3H, ⁴J=1.3 Hz, H-1 or H-4), 2.61 (d, 3H, ⁴J=1.3 Hz, H-1 or H-4), 3.60-3.68 (m, 2H, H-5 or H-6), 3.81-3.88 (m, 1H, H-5 or H-6), 4.08-4.15 (m, 1H, H-5 or H-6), 6.35 (“s”, 1H, H-8), 6.50 (q, 1H, ⁴J=1.3 Hz, H-2 or H-3), 6.73 (q, 1H, ⁴J=1.3 Hz, H-2 or H-3), 7.06 (ddd, 1H, ³J=8.7 Hz, ³J=6.7 Hz, ⁴J=1.1 Hz, H-10 or H-11), 7.18 (ddd, 1H, ³J=8.6 Hz, ³J=6.7 Hz, ⁴J=1.0 Hz, H-10 or H-11), 7.39 (dt, 1H, ³J=8.6 Hz, J=1.1 Hz, H-12), 7.88 (dq, 1H, ³J=8.7 Hz, J=1.0 Hz, H-9).

### 1-(2-Methyl-inden-1-yl)-2-(2,5-dimethyl-cyclopenta[1,2-b;4,3-b′]dithiophen-7-yl)-ethane

To 0.99 g (7.60 mmol) of 2-Methylindene in 20 ml of dry THF were added 4.80 ml n-butyllithium (7.60 mmol/1.6M in hexane) under argon and ice cooling. The solution was stirred for 2 h at rt and dropped to a solution of 2.90 g of trifluoromethanesulfonic acid 2-(2,5-dimethyl-7H-cyclopenta[1,2-b; 4,3-b′]dithiophen-7-yl)-ethyl ester in 20 ml of dry THF cooled to −60° C. The mixture was stirred at rt for 90 min, washed with sat. NH₄Cl and concentrated. The resulting redbrown oil was purified by column chromatography on silica (hexane:dichloromethane 10:1) to get 1.10 g (3.03 mmol/40%) of a yellow oil (mixture of double bond isomers).

GCMS (EI): m/z=362 (M⁺).

¹H-NMR (500.1 MHz, CD₂Cl₂, δ=1.27 (dddd, 1H, ²J=13.4 Hz, ³J=12.2 Hz, ³J=6.7 Hz, ³J=4.5 Hz, H-4-b), 1.42 (dddd, 1H, ²J=13.5 Hz, ³J=12.3 Hz, ³J=6.8 Hz, ³J=4.4 Hz, H-4a), 1.92 (dddd, 1H, ²J=13.6 Hz, ³J=12.3 Hz, ³J=5.5 Hz, ³J=4.4 Hz, H-5b), 2.04 (dd, 3H, ⁴J=1.6 Hz, ⁴J=0.6 Hz, H-6), 2.13 (dddd, 1H, ²J=13.7 Hz, ³J=12.2 Hz, ³J=4.6 Hz, ³J=4.6 Hz, H-5a), 2.52 (d, 3H, ⁴J=1.3 Hz, H-1b), 2.52 (d, 3H, ⁴J=1.3 Hz, H-1a), 3.32-3.35 (m, 1H, H-12), 3.73 (t, 1H, ³J=6.8 Hz, H-3), 6.45 (dq, 1H, ⁴J=1.7 Hz, ⁴J=1.7 Hz, H-7), 6.75 (q, 2H, ⁴J=1.2 Hz, H-2), 7.08 (ddd, 1H, ³J=7.2 Hz, ³J=7.0 Hz, ⁴J=1.8 Hz, H-9), 7.15-7.20 (m, 2H, H-10 and H-11), 7.31 (dt, 1H, ³J=7.4 Hz, ⁴J=0.9 Hz, H-8).

¹H-NMR (500.1 MHz, CD₂Cl₂, db isomer 2): δ=1.95-1.99 (m, 2H, H-4 or H-5), 2.07 (s, 3H, H-6), 2.56 (d, 3H, ⁴J=1.1 Hz, H-1b), 2.56 (d, 3H, ⁴J=1.1 Hz, H-1a), 2.61-2.64 (m, 2H, H-4 or H-5), 3.26-3.27 (m, 2H, H-7), 3.95 (t, 1H, ³J=6.6 Hz, H-3), 6.80 (q, 2H, ⁴J=1.2 Hz, H-2), 7.08 (ddd, 1H, ³J=7.4 Hz, ³J=6.2 Hz, ⁴J=2.3 Hz, H-9), 7.20-7.25 (m, 2H, H-10 and H-11), 7.35 (dt, 1H, ³J=7.3 Hz, ⁴J=1.0 Hz, H-8).

### Trifluoromethanesulfonic 2-(2,5-dimethyl-7H-cyclopenta[1,2-b;4,3-b′]dithiophen-7-yl)-ethyl ester

To a solution of 1.90 g (7.60 mmol) of 2-(2,5-Dimethyl-cyclopenta[1,2-b;4,3-b′]dithiophen-7-yl)-ethanol and 0.7 ml (7.70 mmol) of pyridine in 20 ml of dry dichloromethane was added a solution of 1.3 ml (7.60 mmol) trifluoromethanesulfonic acid anhydride under argon and ice cooling. The resulting suspension was stirred for 20 min at rt, washed with ice cold water and concentrated. The obtained dark oil was immediately used.

¹H-NMR (500.1 MHz, CD₂Cl₂, MIS 260): δ=2.31 (dt, 2H, ³J=6.7 Hz, ⁴J=6.7 Hz, H-4), 2.54-2.54 (m, 6H, H-1), 4.03 (t, 1H, ³J=6.7 Hz, H-3), 4.60 (t, 2H, ³J=6.7 Hz, H-5), 6.79 (q, 2H, ⁴J=1.2 Hz, H-2).

### 2-(2,5-Dimethyl-cyclopenta[1,2-b;4,3-b′]dithiophen-7-yl)-ethanol

To a solution of 5.40 g (26.0 mmol) of 2,5-Dimethyl-cyclopenta[1,2-b;4,3-W]dithiophene in 50 ml of dry toluene were added 16.3 ml (26.0 mmol/1,6M in diethylether) methyllithium under argon and ice cooling. After stirring for 30 min at rt a solution of lithium-2-chloroethanolate in 20 ml of dry toluene, freshly prepared by addition of 16.3 ml (26.0 mmol/1.6M in diethylether) methyllithium to 2-chloroethanol in 20 ml of dry toluene under argon and ice cooling, was added. The resulting mixture was stirred for 3 h at rt, washed with sat. NH₄Cl and with 15 ml 2M HCl and concentrated. Purification of the obtained orange residue by column chromatography on silica (dichloromethane+2% methanol) leads to 4.40 g (17.6 mmol/67%) of the product.

GCMS (EI): m/z=250 (M⁺).

¹H-NMR (500.1 MHz, CD₂Cl₂, MIS 252): δ=1.45 (t, 1H, ⁴J=5.3 Hz, OH), 1.97 (dt, 2H, ³J=7.2 Hz, ³J=6.3 Hz, H-4), 2.53-2.53 (m, 6H, H-1), 3.84 (dt, 2H, ⁴J=5.2 Hz, ³J=6.4 Hz, H-5), 4.01 (t, 1H, ³J=7.2 Hz, H-3), 6.78 (q, 2H, ⁴J=1.2 Hz, H-2).

### Ethylene(2,4,7-trimethyl-indenyl)-(2,5-Dimethyl-cyclopenta[1,2-b;4,3-b′]dithiophene-7-yl)-ZrCl₂ (A2)

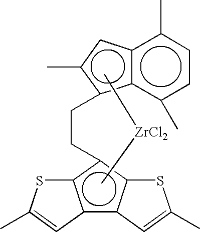

To 0.50 g (1.28 mmol) of 1-(2,4,7-trimethyl-inden-1-yl)-2-(2,5-dimethyl-7H-cyclopenta[1,2-b;4,3-b′]dithiophen-7-yl)-ethane in 20 ml of dry diethylether were added 1.02 ml n-butyllithium (2.56 mmol/2.5M in hexane) under argon and ice cooling. The resulting suspension was stirred for 4 h at rt. To this suspension was added a zirconiumtetrachloride-2THF complex, freshly prepared by addition of 0.18 g (2.56 mmol) of THF to a solution of 0.30 g (1.28 mmol) of zirconiumtetrachloride in 5 ml of dry pentane under argon and ice cooling and stirring for 30 min. The orange suspension was stirred over night at rt and concentrated. The residue was elutriated with 15 ml of dry THF for 1 h. The suspension was filtered, the filter cake was washed with 2 ml of dry THF and dried to get the complex as an orange powder (168 mg/0.31 mmol/24%).

¹H-NMR (500.1 MHz, CD₂Cl₂, MIS 633): δ=2.24 (s, 3H, H-9 or H-12), 2.43 (d, 3H, ⁴J=1.3 Hz, H-1 or H-4), 2.50 (s, 3H, H-7), 2.61 (d, 3H, ⁴J=1.3 Hz, H-1 or H-4), 2.91 (s, 3H, H-9 or H-12), 3.67-3.76 (m, 1H, H-5 or H-6), 3.82-3.92 (m, 3H, H-5 or H-6), 6.45 (s, 1H, H-8), 6.60 (q, 1H, ⁴J=1.3 Hz, H-2 or H-3), 6.69-6.71 (m, 1H, H-10 or H-11), 6.70 (q, 1H, ⁴J=1.3 Hz, H-2 or H-3), 6.79 (dq, 1H, ³J=6.9 Hz, ⁴J=1.1 Hz, H-10 or H-11).

### 1-(2,4,7-Trimethyl-inden-1-yl)-2-(2,5-dimethyl-cyclopenta[1,2-b;4,3-b′]dithiophen-7-yl)ethane

To 0.80 g (5.00 mmol) of 2,4,7-trimethylindene in 15 ml of dry THF were added 3.20 ml methyllithium (5.00 mmol/1.6M in diethylether) under argon and ice cooling. The solution was stirred for 3 h at rt and dropped to a solution of 2.10 g (5.00 mmol) of trifluoromethanesulfonic acid 2-(2,5-dimethyl-cyclopenta[1,2-b;4,3-b′]dithiophen-7-yl)-ethyl ester in 15 ml of dry THF cooled to −50° C. The mixture was stirred at rt over night, washed with sat. NH₄Cl and concentrated. The resulting dark oil was purified by column chromatography on silica (hexane:dichloromethane 10:1) to get 1.0 g (2.56 mmol 51%) of a solid (mixture of double bond isomers).

GCMS (EI): m/z=390 (M⁺).

¹H-NMR (500.1 MHz, CD₂Cl₂, MIS 264): δ=1.04 (dddd, 1H, ²J=13.4 Hz, ³J=12.9 Hz, ³J=6.5 Hz, ³J=4.5 Hz, H-4b), 1.23 (dddd, 1H, ²J=13.4 Hz, ³J=12.9 Hz, ³J=6.3 Hz, ³J=4.1 Hz, H-4a), 1.87 (dddd, 1H, ²J=13.7 Hz, ³J=12.7 Hz, ³J=3.9 Hz, ³J=3.9 Hz, H-5b), 2.02 (d, 3H, ⁴J=2.1 Hz, H-6), 2.19 (dddd, 1H, ²J=13.6 Hz, ³J=12.9 Hz, ³J=5.0 Hz, ³J=4.9 Hz, H-5a), 2.28 (s, 3H, H-8 or H-11), 2.30 (s, 3H, H-8 or H-11), 2.52 (d, 3H, ⁴J=1.3 Hz, H-1b), 2.52 (d, 3H, ⁴J=1.3 Hz, H-1a), 3.38 (tm, 1H, ³J=4.4 Hz H-12), 3.71 (t, 1H, ³J=6.4 Hz, H-3), 6.54 (dq, 1H, ⁴J=1.6 Hz, ⁴J=1.6 Hz, H-7), 6.74 (q, 1H, ⁴J=1.1 Hz, H-2b), 6.74 (q, 1H, ⁴J=1.1 Hz, H-2a), 6.75 (d, 1H, ³J=7.6 Hz, H-9 or H-10), 6.89 (d, 1H, ³J=7.6 Hz, H-9 or H-10).

### Ethylene(2-methyl-5,6,7-trihydro-s-indacen-1-yl)-(2,5-dimethyl-cyclopenta[1,2-b;4,3-b′]dithiophene-7-yl)-ZrCl₂ (A3)

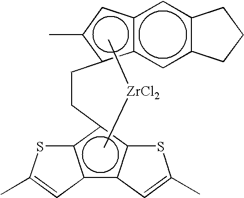

To 0.50 g (1.24 mmol) of 1-(2-methyl-5,6,7-trihydro-s-indacen-1-yl)-2-(2,5-dimethyl-cyclopenta[1,2-b;4,3-b′]dithiophen-7-yl)-ethane in 10 ml of dry diethylether and 10 ml of dry toluene were added 0.99 ml n-butyllithium (2.48 mmol/2.5M in hexane) under argon and ice cooling. The resulting white suspension was stirred for 3 h at rt. To this suspension was added a suspension of 0.29 g (1.24 mmol) of zirconiumtetrachloride in 10 ml of dry toluene. The dark brown suspension was stirred over night at rt and filtered. The organic layer was concentrated, washed four times with 3 ml of dry diethylether in each case and dried to get 125 mg (0.22 mmol/22%) of a beige powder.

¹H-NMR (500.1 MHz, CD₂Cl₂, MIS 561): δ=2.00-2.06 (m, 2H, H-11), 2.32 (s, 3H, H-7), 2.40 (d, 3H, ⁴J=1.3 Hz, H-1 or H-4), 2.60 (d, 3H, ⁴J=1.3 Hz, H-1 or H-4), 2.86-3.00 (m, 4H, H-10 and H-12), 3.57-3.62 (m, 2H, H-5 or H-6), 3.79-3.86 (m, 1H, H-5 or H-6), 4.11-4.18 (m, 1H, H-5 or H-6), 6.22 (s, 1H, H-8), 6.48 (q, 1H, ⁴J=1.3 Hz, H-2 or H-3), 6.71 (q, 1H, ⁴J=1.3 Hz, H-2 or H-3), 7.20 (dt, 1H, ⁴J=1.3 Hz, ⁴J=1.4, H-9), 7.67 (“s”, 1H, H-13).

### 1-(2-Methyl-5,6,7-trihydro-s-indacen-1-yl)-2-(2,5-dimethyl cyclopenta[1,2-b;4,3-b′]dithiophen-7-yl)-ethane

To 3.20 g (18.4 mmol) of 6-methyl-1,2,3,5-tetrahydro-s-indacene in 30 ml of dry THF were added 11.5 ml methyllithium (18.4 mmol/1.6M in diethylether) under argon and ice cooling. The solution was stirred for 2 h at rt and dropped to a solution of 7.30 g of trifluoromethanesulfonic 2-(2,5-dimethyl-7H-cyclopenta[1,2-b;4,3-W]dithiophen-7-yl)-ethyl ester in 50 ml of dry THF cooled to −60° C. The mixture was stirred at rt over night, washed with sat. NH₄Cl and concentrated. The resulting redbrown oil was purified by column chromatography on silica (hexane:dichloromethane 6:1) to get 2.40 g (5.96 mmol/32%) of a yellow solid (mixture of double bond isomers 3:1).

GCMS (EI): m/z=402 (M⁺).

¹H-NMR (500.1 MHz, CD₂Cl₂: δ=1.92-1.97 (m, 2H, H-4), 2.04 (s, 3H, H-6), 2.07 (quint., 2H, ³J=7.4 Hz, H-10), 2.55 (d, 3H, ⁴J=1.1 Hz, H-1b), 2.55 (d, 3H, ⁴J=1.1 Hz, H-1a), 2.59-2.62 (m, 2H, H-5), 2.88 (t, 2H, ³J=7.4 Hz, H-9 or H-11), 2.90 (t, 2H, ³J=7.4 Hz, H-9 or H-11), 3.19-3.20 (m, 2H, H-7), 3.94 (t, 1H, ³J=6.7 Hz, H-3), 6.80 (q, 2H, ⁴J=1.2 Hz, H-2), 7.07 (s, 1H, H-8 or H-12), 7.20-7.20 (m, 1H, H-8 or H-12).

### Dimethylsilyl-(2-methyl-indenyl)-(2,5-Dimethyl-7H-cyclopenta[1,2-b; 4,3-b′]dithiophenyl)-ZrCl₂ (C1) and dimethylsilyl-(2,4,7-trimethyl-indenyl)-(2,5-Dimethyl-7H-cyclopenta[1,2-b;4, 3-13′]dithiophenyl)-ZrCl₂ (C2) have been synthesized according to WO01/047939.

### Dimethylsilyl(2-methyl-5,6,7-trihydro-s-indacenyl)-(2,5-dimethyl-7H-cyclopenta[1,2-b;4,3-b′]dithiophenyl)-ZrCl₂ (C3)

### Synthesis of chlorodimethyl(2-methyl-1,5,6,7-tetrahydro-s-indacen-1-yl)silane

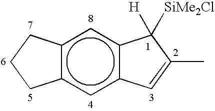

10.5 g of 6-methyl-1,2,3,5-tetrahydro-s-indacene (61.67 mmol) were dissolved at room temperature under nitrogen atmosphere in 30 mL of Et₂O in a 100 mL Schlenk flask obtaining a brown-orange solution. The latter was cooled to 0÷4° C. with an ice bath and added under stirring of 25.0 mL of a 2.5 M BuLi solution in hexane (62.50 mmol, n-BuLi/indacene=1.01/1.00). At the end of the addition additional 10 mL of Et₂0 and 10 mL of THF were slowly added at 0÷4° C. in order to improve the stirring of the resulting mixture. Then it was allowed to reach room temperature and stirred for 3 h at this temperature obtaining a dark brown suspension. Then the reaction mixture was cooled again to 0÷4° C. and slowly added to a solution of 9.52 g of Me₂SiCl₂ (Aldrich 99%, 73.8 mmol, 1.2 eq) in 20 mL of Et₂O, previously cooled to 0° C. too. At the end of the addition, the reaction mixture was allowed to reach room temperature and stirred for 16 h with final formation of a yellow-brown suspension. The latter was dried at 50° C. in vacuum giving a sticky orange-brown solid, which was added of 85 mL of anhydrous toluene: the resulting suspension was stirred for 45 min at room temperature and then filtered under nitrogen atmosphere on a G3 frit in order to remove LiCl as white solid. The filtrate resulted to be a clear orange-brown solution, which was dried at 60° C. in vacuum obtaining 13.75 g of the target product. The latter was used as such in the next step without further purification. Contained yield was 84.8%.

### Synthesis of 1-(2-methyl-1,5,6,7-tetrahydro-s-indacenyl)-7-(2,5-dimethyl-cyclopenta[1,2-b:4,3-b′]-dithiophene) dimethylsilane

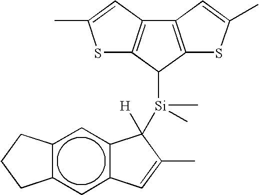

2.54 g of 2,5-dimethyl-7H-cyclopenta[1,2-b:4,3-b′]-dithiophene (12.31 mmol) were suspended at room temperature under nitrogen atmosphere in 60 mL of Et₂O in a 250 mL Schlenk flask; the brown suspension was cooled to 0÷4° C. with an ice bath and added under stirring of 5.2 mL of a 2.5 M n-BuLi solution in hexane (13.00 mmol, n-BuLi/dithiophene=1.06/1.00). At the end of the addition, the reaction mixture was stirred at 0÷4° C. for 1.5 h with final formation of a black suspension. Then it was slowly added to an orange solution of 3.26 g of chlorodimethyl(2-methyl-1,5,6,7-tetrahydro-s-indacen-1-yl)silane (12.40 mmol, silane/dithiophene=1.01:1.00) in 40 mL of THF, previously cooled to 0÷4° C. too. At the end of the addition, the reaction mixture was kept at 0÷4° C. for 1 h, then allowed to reach room temperature and stirred for 24 h, with final formation of a black suspension. The solvents were removed at 40° C. in vacuum and the resulting sticky black solid was taken up into 100 mL of toluene. The suspension was stirred for few hours at room temperature and then filtered over a G3 frit in order to remove LiCl as solid. The filtrate was dried at 50° C. for 2 h in vacuum, obtaining 5.54 g of a black solid, which resulted to be by NMR analysis the desired product, contaminated by starting dithiophene. Nevertheless this crude product was used as such in the next step without further purification. The purity in 1-(2-methyl-1,5,6,7-tetrahydro-s-indacenyl)-7-(2,5-dimethyl-cyclopenta[1,2-b:4,3-b′]-dithiophene) dimethylsilane was ca. 80% wt.

### Synthesis of dimethylsilanediyl[1-(2-methyl-1,5,6,7-tetrahydro-s-indacenyl)-7-(2,5-dimethyl-cyclopenta[1,2-b:4,3-b′]-dithiophene)]zirconium dichloride (C3)

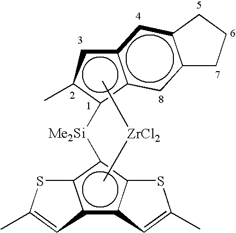

5.54 g of 1-(2-methyl-1,5,6,7-tetrahydro-s-indacenyl)-7-(2,5-dimethyl-cyclopenta[1,2-b:4,3-b′]-dithiophene) dimethylsilane (12.80 mmol), prepared as above described, were dissolved at room temperature under nitrogen atmosphere in 80 mL of ethyl ether in a 250 mL Schlenk flask obtaining a black solution. The latter was cooled to 0÷4° C. with an ice bath and added under stirring of 10.5 mL of a 2.5 M n-BuLi solution in hexane (26.25 mmol, n-BuLi/dithiophene=2.05/1.00) giving a black suspension, which was stirred at 0÷4° C. for 15 min and at room temperature for 1 h. Then it was cooled again to 0-4° C. and added slowly to a slurry of 2.94 g of ZrCl₄ (12.62 mmol) in 80 mL of anhydrous toluene, previously cooled to 0÷4° C. too. The resulting reaction mixture was kept at 0÷4° C. for 20 min and then allowed to reach room temperature. After 5 h 30′ stirring at room temperature the mixture resulted to be a brown suspension: the solvents were removed in vacuum until reaching a left volume of 50 mL. Thus the reaction mixture was left at rest overnight and the day after filtered on a G3 frit: the filtrate containing impurities and traces of the complex was discarded, while the residue containing LiCl and the metallocene was dried yielding an orange-brown sticky solid (ca. 6.9 g). The latter was washed under stirring at room temperature on the frit with 40 mL of ethyl ether and finally dried under vacuum, giving 4.32 g of a light orange powder, which contains the target complex and 12.9% wt. of LiCl. Yield based on zirconium was 50.3%.

¹H-NMR (CD₂Cl₂, δ in ppm): 1.17 (s, 3H, Si-CH₃); 1.34 (s, 3H, Si-CH₃); 2.00 (quintet, 2H, J=7.49 Hz, CH₂); 2.34 (s, 3H, CH₃); 2.44 (d, 3H, J=1.35 Hz, CH₃); 2.60 (d, 3H, J=1.35 Hz, CH₃); 2.66-3.00 (m, 4H, CH₂); 6.33 (q, 1H, J=1.35 Hz, CH); 6.79 (q, 1H, J=1.35 Hz, CH); 6.68 (s, 1H, CH); 7.26 (s, 1H, CH); 7.49 (s, 1H, CH).

### Catalyst Systems:

Al(i-Bu)₃ (TIBA) and methylalumoxane (MAO, Albemarle 30% wt/wt in toluene) were used as received

### C2/MAO:TIBA 2:1 (Al/Zr=400): CC2

14.4 ml of TIBA/isododecane solution (110 g/l) were mixed with 3.5 ml of MAO/toluene solution to obtain a MAO/TIBA molar ratio of 2:1. The solution was stirred for 30 minutes at room temperature. Then, 32.35 mg of C2 (MW 580.81) were dissolved in the solution.

The solution did not show any trace of residual solid.

The final solution was diluted with 7.5 mL of toluene to reach a concentration of 100 g/l (1.27 mg C2/ml).

### A1/MAO:TIBA 2:1 (Al/Zr=400): CA1

14.4 ml of TIBA/isododecane solution (110 g/l) were mixed with 3.5 ml of MAO/toluene solution to obtain a MAO/TIBA molar ratio of 2:1. The solution was stirred for 30 minutes at room temperature. Then, 31.5 mg of A1 (MW 522.67) were dissolved in the solution.

The solution did not show any trace of residual solid.

The final solution was diluted with 7.6 ml of toluene to reach a concentration of 100 g/l (1.24 mg Al/ml).

### C3/MAO:TIBA 2:1 (Al/Zr=400): CC3

14.4 ml of TIBA/isododecane solution (110 g/l) were mixed with 3.5 ml of MAO/toluene solution to obtain a MAO/TIBA molar ratio of 2:1. The solution was stirred for 30 minutes at room temperature. Then, 40.8 mg of C3 (MW 592.84) were dissolved in the solution.

The solution did not show any trace of residual solid.

The final solution was diluted with 7.5 ml of toluene to reach a concentration of 100 g/L (1.40 mg C3/ml).

### C1/MAO:TIBA 2:1(Al/Zr=400): CC1

14.4 ml of TIBA/isododecane solution (110 g/l) were mixed with 3.5 ml of MAO/toluene solution to obtain a MAO/TIBA molar ratio of 2:1. The solution was stirred for 30 minutes at room temperature. Then, 39.96 mg of C1 (MW 552.76) were dissolved in the solution.

The solution did not show any trace of residual solid.

The final solution was diluted with 7.5 ml of toluene to reach a concentration of 100 g/l (1.3 mg C1/ml).

### A3/MAO:TIBA 2:1 (Al/Zr=400): CA3

14.4 ml of TIBA/isododecane solution (110 g/l) were mixed with 3.5 ml of MAO/toluene solution to obtain a MAO/TIBA molar ratio of 2:1. The solution was stirred for 30 minutes at room temperature. Then, 32.35 mg of A3 (MW 562.74) were dissolved in the solution.

The solution did not show any trace of residual solid.

The final solution was diluted with 7.5 ml of toluene to reach a concentration of 100 g/l (1.33 mg A3/ml).

### A2/MAO:TIBA 2:1 (Al/Zr=400): CA2

14.4 ml of TIBA/isododecane solution (110 g/l) were mixed with 3.5 ml of MAO/toluene solution to obtain a MAO/TIBA molar ratio of 2:1. The solution was stirred for 30 minutes at room temperature. Then, 32.26 mg of A2 (MW 550.72) were dissolved in the solution.

The solution did not show any trace of residual solid.

The final solution was diluted with 7.6 ml of toluene to reach a concentration of 100 g/l (1.30 mg A2/ml).

### Polymerization Tests.

The 4.41 jacketed stainless-steel autoclave, equipped with a magnetically driven stirrer and a 35-ml stainless-steel vial and connected to a thermostat for temperature control, was previously purified by washing with an Al(i-Bu)₃ solution in hexane and dried at 50° C. in a stream of nitrogen.

6 mmol of Al(i-Bu)₃ (as a 100 g/l solution in hexane) and 1350 g of 1-butene were charged at room temperature. The autoclave was then thermostated at the polymerization temperature, 70° C., corresponding at a pressure of 10 bar-g.

An amount of the solution of catalyst system of 1.5 ml containing the catalyst/cocatalyst mixture was injected in the autoclave by means of 4 ml of cyclohexane through the stainless-steel vial. A constant temperature was maintained for 60 minutes.

The autoclave filled with 1 l cyclohexane in order to dilute the solution and the bottom discharge valve was opened and the copolymer was discharged into a heated steel tank containing water at 70° C. The tank heating was switched off and a flow of nitrogen at 0.5 bar-g was fed. After cooling at room temperature, the steel tank was opened and the wet polymer collected. The wet polymer was dried in an oven under reduced pressure at 70° C. The polymerization results are reported in table 1

TABLE 1

| | | Polymerization |
|---|---|---|
| | Catalyst | activity |
| run | system | kgₚₒₗ/g_{metallocene}/h |
| 1 | CA1 | 200 |
| 2* | CC1 | 35 |
| 3 | CA2 | 163 |
| 4* | CC2 | 60 |
| 5 | CA3 | 150 |
| 6* | CC3 | 55 |

*comparative

From table 1 clearly results that the ethylene bridged metallocene compounds show an activity more than 3 times higher with respect to the analogues silyl bridged compounds.

1. A metallocene compound of formula (I): 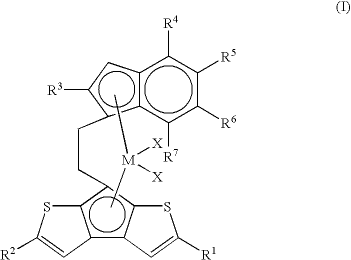
wherein
M is an atom of a transition metal selected from those belonging to group 3, 4, or to the lanthanide or actinide groups in the Periodic Table of the Elements;
X, equal to or different from each other, is a hydrogen atom, a halogen atom, an R, OR, OSO₂CF₃, OCOR, SR, NR₂ or PR₂ group wherein R is a linear or branched, cyclic or acyclic, C₁-C₄₀-alkyl, C₂-C₄₀ alkenyl, C₂-C₄₀ alkynyl, C₆-C₄₀-aryl, C₇-C₄₀-alkylaryl or C₇-C₄₀-arylalkyl radical; optionally containing heteroatoms belonging to groups 13-17 of the Periodic Table of the Elements; or two X groups can be joined together to form a group OR′O wherein R′ is a C₁-C₂₀-alkylidene, C₆-C₂₀-arylidene, C₇-C₂₀-alkylarylidene, or C₇-C₂₀-arylalkylidene radical;
R¹ and R², equal to or different from each other, are C₁-C₄₀ hydrocarbon radicals optionally containing heteroatoms belonging to groups 13-17 of the Periodic Table of the Elements;
R³ is a C₁-C₄₀ hydrocarbon radical optionally containing heteroatoms belonging to groups 13-17 of the Periodic Table of the Elements;
R⁴, R⁵, R⁶ and R⁷, equal to or different from each other, are hydrogen atoms or C₁-C₄₀ hydrocarbon radicals optionally containing heteroatoms belonging to groups 13-17 of the Periodic Table of the Elements or groups among R⁴, R⁵, R⁶ and R⁷ can also be joined to form a from 4 to 7 membered ring.

2. The metallocene according to claim 1 having formula (IIa):

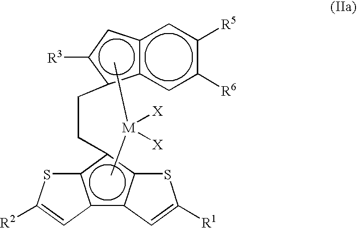

3. The metallocene according to claim 1 having formula (IIb): 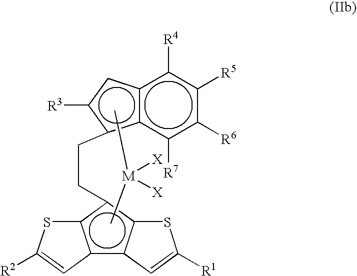
wherein R⁴ and R⁷ equal to or different from each other, are hydrogen atoms, C₁-C₂₀ alkyl, C₆-C₂₀-aryl, C₇-C₂₀-alkylaryl or C₇-C₂₀-arylalkyl radicals; with the proviso that at least one between R⁴ and R⁷ is different from hydrogen atom.

4. The metallocene according to claim 1 having formula (IIc): 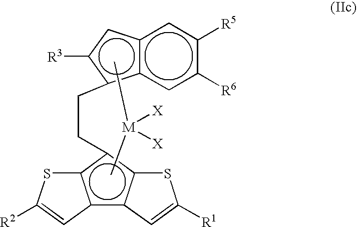
wherein R⁵ and R⁶, equal to or different from each other, are C₁-C₂₀ alkyl radicals optionally containing heteroatoms belonging to groups 15-16 of the periodic table, or R⁵ and R⁶ can be joined to form a 5-6 membered ring, said ring can be aliphatic or aromatic and can contain heteroatoms belonging to groups 15-16 of the periodic table; said ring can further bear C¹—C¹⁰ alkyl substituents.

5. The metallocene according to claim 1 having formula (IId): 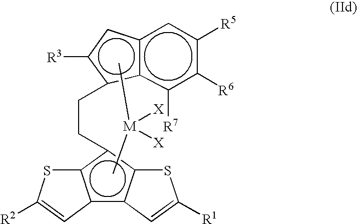
wherein R⁴ and R⁷, equal to or different from each other, are C₁-C₂₀ alkyl, C₆-C₂₀-aryl, C₇-C₂₀-alkylaryl or C₇-C₂₀-arylalkyl radicals.

6. A catalyst system for the polymerization of olefins obtained by contacting:
a) a metallocene compound of formula (I): 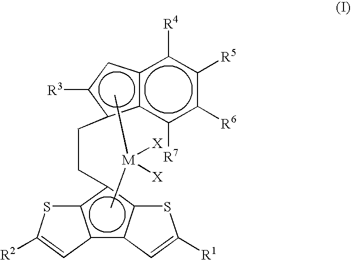
wherein
M is an atom of a transition metal selected from those belonging to group 3, 4, or to the lanthanide or actinide groups in the Periodic Table of the Elements;
X, equal to or different from each other, is a hydrogen atom, a halogen atom, an R, OR, OSO₂CF₃, OCOR, SR, NR₂, or PR₂, group wherein R is a linear or branched, cyclic or acyclic, C₁-C₄₀-alkyl, C₂-C₄₀ alkenyl, C₂-C₄₀ alkynyl, C₆-C₄₀-aryl, C₇-C₄₀-alkylaryl or C₇-C₄₀-arylalkyl radical; optionally containing heteroatoms belonging to groups 13-17 of the Periodic Table of the Elements; or two X groups can be joined together to form a group OR′O wherein R′ is a C₁-C₂₀-alkylidene, C₆-C₂₀-arylidene, C₇-C₂₀-alkylarylidene, or C₇-C₂₀-arylalkylidene radical;
R¹ and R², equal to or different from each other, are C₁-C₄₀ hydrocarbon radicals optionally containing heteroatoms belonging to groups 13-17 of the Periodic Table of the Elements;
R³ is a C₁-C₄₀ hydrocarbon radical optionally containing heteroatoms belonging to groups 13-17 of the Periodic Table of the Elements;
R⁴, R⁵, R⁶ and R⁷, equal to or different from each other, are hydrogen atoms or C₁-C₄₀ hydrocarbon radicals optionally containing heteroatoms belonging to groups 13-17 of the Periodic Table of the Elements or groups among R⁴, R⁵, R⁶ and R⁷ can also be joined to form a from 4 to 7 membered ring;
b) at least an alumoxane or a compound that forms an alkylmetallocene cation; and
c) optionally an organo aluminum compound.

7. A process for preparing an alpha-olefin polymer comprising contacting under polymerization conditions at least one alpha-olefin of formula CH₂═CHA wherein A is hydrogen or a C₁-C₂₀ alkyl radical, in the presence of a catalyst system as described in claim 6.

8. The process according to claim 7 wherein 1-butene and optionally ethylene, propylene or one or more alpha olefins of formula CH₂═CHA¹, wherein A¹ is a C₃-C₂₀ alkyl radical are contacted to produce 1-butene homo or copolymers.

9. A ligand of formula (III): 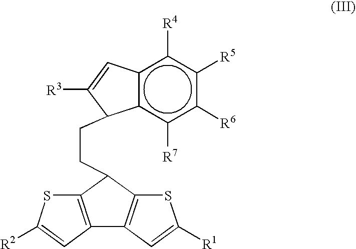
or its double bond isomer
wherein R ¹ and R², equal to or different from each other, are C₁-C₄₀ hydrocarbon radicals optionally containing heteroatoms belonging to groups 13-17 of the Periodic Table of the Elements;
R³ is a C₁-C₄₀ hydrocarbon radical optionally containing heteroatoms belonging to groups 13-17 of the Periodic Table of the Elements;
R⁴, R⁵, R⁶ and R⁷, equal to or different from each other, are hydrogen atoms or C₁-C₄₀ hydrocarbon radical optionally containing heteroatoms belonging to groups 13-17 of the Periodic Table of the Elements or groups among R⁴, R⁵, R⁶ and R⁷ can also be joined to form a from 4 to 7 membered ring.

10. A process for the preparation of the ligand of formula (III) of claim 9 comprising the following steps:
i) contacting the moiety of formula (IVa) 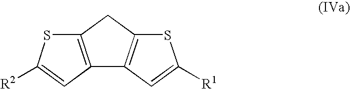
wherein R¹ and R², equal to or different from each other, are C₁-C₄₀ hydrocarbon radicals optionally containing heteroatoms belonging to groups 13-17 of the Periodic Table of the Elements,
with a base selected from the group consisting of metallic sodium and potassium, sodium and potassium hydroxide and an organolithium compound, wherein the molar ratio between the compound of the formula (II) and said base is at least 1:1;
ii) contacting the reaction product of step i) with a compound of formula (IVb): 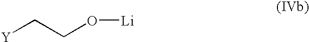
wherein Y is a halogen atom, and a compound that donates a proton, to form a compound of formula (IVc): 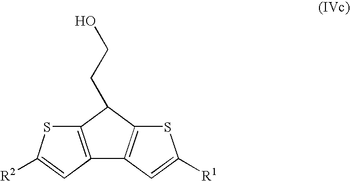
iii) contacting the compound of formula (IVc) with trifluoromethanesulfonic acid anhydride in the presence of a weak broensted base to form the product of formula (IVd): 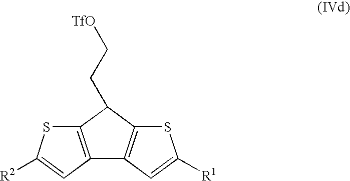
wherein TfO is the trifluoromethanilsulfonate residue;
iv) contacting the product (IVd) with a salt of formula (IVe): 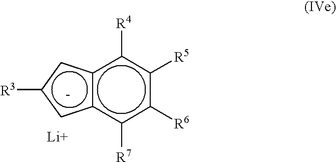
to obtain the final product.

11. The process of claim 10, wherein the organolithium compound has formula LiR^{a} wherein R^{a} is a C₁-C₄₀ hydrocarbon group.

12. The process claim 11, wherein R^{a} is a C₁-C₄₀-alkyl, C₆-C₄₀-aryl, C₇-C₄₀-alkylaryl or C₇-C₄₀-arylalkyl radical.

13. The process of claim 10, wherein the compound that donates a proton is water or HCl.

14. The process of claim 10, wherein the weak broensted base is pyridine or a tertiary amine.
